(19) United States
(12) Patent Application Publication
(10) Pub. No.: US 2003/0028910 A1
(43) Pub. Date: Feb. 6, 2003

(54) Non-human transgenic animal whose germ cells and somatic cells contain a knockout mutation in DNA encoding orphan nuclear receptor ERRalpha

(76) Inventors: Vincent Giguere, Outremont (CA); Robert Sladek, Montreal (CA); Jiang-Ming Luo, Marshfield, WI (US)

Correspondence Address: MORRISON & FOERSTER LLP, 755 PAGE MILL RD, PALO ALTO, CA, 94304-1018, US

(21) Appl. No.: 09/925,720

(22) Filed: Aug. 8, 2001

Related U.S. Application Data
(60) Provisional application No. 60/119,024, filed on Feb. 8, 1999.

(30) Foreign Application Priority Data
Feb. 8, 2000 (CA) PCT/CA00/00145

(51) Int. Cl. A01K 67/027

(52) U.S. Cl. 800/18

(57) ABSTRACT

The present invention relates to a transgenic non-human mammal whose germ cells and somatic cells contain a knockout mutation in DNA encoding orphan nuclear receptor ERRα. More specifically, the invention relates to knockout mice whose germ cells and somatic cells contain a knockout mutation in DNA encoding orphan nuclear receptor ERRα. The invention further relates to such knockout non-human mammals which express an ERRα gene which is different from the endogenous gene which was disrupted. In a particular embodiment, the invention relates to a transgenic mouse having its endogenous ERRα gene disrupted and expressing human ERRα. As well, the invention relates to cell lines in which ERRα activity (and/or level) has been inactivated or augmented. The invention further relates to uses and methods of the transgenic animals of the present invention to select agents which modulate the expression and/or activity of ERRα.

### FIELD OF THE INVENTION

The present invention relates to a transgenic non-human animal whose germ cells and somatic cells contain a knockout mutation in DNA encoding orphan nuclear receptor ERRα. More particularly, the invention relates to a non-human transgenic mammal whose germ cells and somatic cells contain a knockout mutation in DNA encoding orphan nuclear receptor ERRα and more specifically to a transgenic mice whose germ cells and somatic cells contain a knockout mutation in DNA encoding orphan nuclear receptor ERRα. In one particular embodiment, mice containing a disruption of both copies of the ERRα gene lack detectable expression of the ERRα protein. The invention further relates to such knockout non-human animals which express an Errα gene which is different from the endogenous gene which was disrupted. In a particular embodiment, the invention relates to a transgenic mouse having its endogenous ERRα gene disrupted and expressing human ERRα. As well, the invention relates to cell lines in which ERRα activity (and/or level) has been inactivated or augmented. The invention further relates to uses and methods of the transgenic animals of the present invention to select agents which modulate the expression and/or activity of ERRα and to agents identified by these methods.

### BACKGROUND OF THE INVENTION

The orphan nuclear receptor estrogen-related receptor α (ERRα) was initially cloned by low stringency screening of human kidney library using the estrogen receptor (ERα) DNA-binding domain as a probe (Giguère et al., 1988). Although ERRα displays significant homology to ERα, it does not bind estrogens in vitro, nor is its transcriptional activity modulated by estrogens (Giguère et al., 1988; Yang et al., 1996). ERRα binds to hormone response elements containing a single consensus half site flanked by the 5′ upstream sequence TNA as well as to consensus estrogen response elements (Bonnelye et al., 1997; Johnston et al., 1997; Sladek et al., 1997). Recent studies performed in vitro have implicated ERRα in a wide variety of physiologic processes, including adipocyte development (Sladek et al., 1997), cellular fatty acid oxidation (Sladek et al., 1997; Vega and Kelly, 1997), bone development (Bonnelye et al., 1997), steroidogenesis (Yang et al., 1998) as well as in thyroid hormone receptor isoform expression (Vanacker et al., 1998). In addition, ERRα has been shown to heterodimerize with ERα in solution and can modulate the estrogen responsiveness of the lactoferrin gene promoter (Yang et al., 1996).

Obesity is a prevalent disorder that often leads to diabetes, cardiovascular disease, and joint disorders. Although the precise mechanism which leads to the development of obesity has yet to be precisely determined, it appears clear that a number of mechanisms, which normally function to maintain homeostasy and normal body weight are involved. Transgenic mice with an induced brown fat deficiency have indicated that this tissue is implicated in the control of the balance of in mice (Lowell et al., Nature 366:740-742, 1993). Further, a correlation between brown adipose tissue dysfunction and obesity and diabetes has been reported (Lowell et al., Supra). Previous studies have demonstrated that ERRα is highly expressed in brown adipose tissue (BAT) during murine development and that the receptor is upregulated during white and brown adipocyte differentiation in vitro (Sladek et al., 1997; Vega and Kelly, 1997). In addition, ERRα has been shown to modulate the activity of the medium chain acyl-coA dehydrogenase (MCAD) promoter, a key regulatory step in the fatty acid β-oxidation pathway (Sladek et al., 1997; Vega and Kelly, 1997). More recently, a transgenic mouse whose germ cells and somatic cells contain a knockout mutation in DNA encoding an endogenous .beta.₃-adrenergic receptor polypeptide, thereby obtaining a mouse having a modest increase in body fat, has been reported (U.S. Pat. No. 5,789,654).

There thus remains a need to identify the physiological function of ERRα in vivo. There also remains a need to better identify which homeostatic mechanism, when disrupted or malfunctioning is implicated in the development of obesity and related diseases. In addition, there remains a need to provide animal models of obesity and related diseases, and model systems which can enable the identification and selection of agents which modulate the pathways implicated in the development of obesity and related diseases. Furthermore, there remains a need to identify a target for the eventual therapy of obesity and related diseases.

The present invention seeks to meet these and other needs. Indeed, in order to identify the precise physiological function of ERRα in vivo, a new strain of mice is herein provided, in which ERRα function has been ablated by homologous recombination in embryonic stem cells. The present invention, in particular, relates to this new strain of mice and to the function of ERRα and related factors in vivo.

The present description refers to a number of documents, the content of which is herein incorporated by reference in their entirety.

### SUMMARY OF THE INVENTION

In general, the present invention relates to ERRα-deficient non-human transgenic mammals. More specifically, the invention relates to a transgenic non-human mammal whose germ cells and somatic cells contain a knockout mutation in DNA encoding the ERRα endogenous orphan nuclear receptor polypeptide. In one embodiment, the transgenic mammal also includes germ cells and somatic cells expressing DNA encoding a non-endogenous ERRα orphan nuclear receptor polypeptide. In a preferred embodiment, the transgenic mammal also includes germ cells and somatic cells expressing DNA encoding human ERRα orphan nuclear receptor polypeptide.

Also in general, the present invention relates to the surprising demonstration that ERRα is implicated in lipogenesis (fatty acid synthesis), fatty acid esterification (triglyceride synthesis), and fatty acid oxydation. Indeed, the ERRα knockout mouse of the present invention displays abnormalities in lipogenesis, fatty acid esterification and fatty acid oxydation. The present invention therefore provides the means to affect these three processes. The knockout mammal of the present invention also demonstrates that the alteration of the activity of ERRα affects weight gain in an animal.

In a further general aspect, the invention relates to ERRα as a target to regulate lipogenesis, fatty acid esterification and fatty acid oxydation in vivo. ERRα, cell lines and animals of the present invention can now be used to screen for regulators of ERRα activity and level. The present invention thus provides the means to identify small diffusible ligands which can modulate the activity of the putative steroid hormone receptor ERRα.

Based on the results presented herein, the inhibition of ERRα activity is relevant to the treatment of glucose metabolism disorders as well as obesity.

Until the present invention, studies of ERRα and its role in cellular physiology were limited to in vitro studies and studies in culture cells, or extracts thereof. Therefore, such studies did not assess the action of ERRα and interacting factors on metabolic pathways dependent on such interactions, which could result in a physiologically significant effect such as, for example, lipogenesis, fatty acid esterification, fatty acid oxydation, and metabolic process controlling energy balance and adiposity in a living animal or preferably in a living mammal.

Prior to the present invention, there had been no demonstration or suggestion that ERRα could have such a significant lipid metabolism, or weight gain, and/or glucose metabolism. In view of the complexity of such physiological pathways, and of the complexity of the transcription machinery operating at estrogen receptor cis-acting sequences (for example, see Sladek et al., 1997) and the fact that ERRα interacts with ERRβ and/or with ERRγ, to modulate transcription promoters comprising such cis-acting elements, there was no teachings or suggestions that a knockout of ERRα could have such a significant impact on the metabolism of an animal. Indeed, in view of the complexity of the interaction of the interacting factors binding to estrogen receptor cis-elements and related cis-elements, to modulate promoter activity of different genes, it could not be reasonably predicted that a knockout of ERRα would not be compensated by other factors which interact therewith (e.g. ERRβ or ERRγ).

In addition, the invention relates to a method of producing a transgenic non-human mammal displaying a lean phenotype the non-human mammal lacking expression of the endogenous ERRα orphan nuclear receptor polypeptide, the method including a disruption of the DNA encoding ERRα, and a selection of progeny whose germ cells and somatic cells contain a knockout mutation in DNA encoding ERRα, thereby yielding a lean non-human transgenic animal. Of course, such lean transgenic animals could also be produced using a reduced amount of ERRα (e.g. using antisense ERRα, for example), as opposed to a total abrogation of its expression. In addition, animals expressing nucleic acid sequence which enables an inhibition of the interaction between ERRα and interacting factors (e.g. cis-response elements and the like) could also be produced.

In a preferred embodiment, the invention relates to transgenic mice homozygous for the ERRα mutation, the mice being viable and fertile but exhibiting lipoatrophy despite normal food intake, fat absorption and metabolic activities. The ERRα-deficient lean mice have higher levels of circulating free fatty acids, and the mutant liver, gut and adipose tissue displayed reduced lipogenesis, fatty acid esterification, and fatty acid oxydation, contributing to the lean phenotype.

Furthermore, the present invention relates to the demonstration that ERRα is required for the regulation of lipogenesis, fatty acid esterification and oxydation and metabolic processes controlling energy balance and adiposity, thereby providing a new target for the development of therapeutics for obesity, fat deposition disorders and related diseases, such as diabetes. The present invention further relates to ERRα as a target for the development of diagnostics for obesity, fat deposition disorders and related diseases.

The present invention further relates to ERRα-deficient non-human mammals as a new model for the investigation of lipid metabolism and associated diseases.

The ERRα-deficient mice of the present invention demonstrate that ERRα is required for the regulation of lipogenesis and metabolic processes controlling energy balance and adiposity and suggest that pharmacologic modulation of ERRα activity may provide means to control obesity in humans. The present invention therefore provides a new model for the investigation of lipid metabolism and associated diseases.

In another aspect, the invention features a method of producing a transgenic non-human mammal capable of expressing a functionally active non endogenous ERRα polypeptide, the non-human mammal lacking expression of the endogenous ERRα polypeptide, the method including: (a) providing a transgenic non-human mammal whose germ cells and somatic cells are deficient in ERRα (i.e. ERRα knockout); (b) introducing a non-endogenous ERRα transgene capable of expressing a ERRα polypeptide, into a cell of the non-human mammal; and (c) obtaining progeny expressing the non-endogenous transgene. In a preferred embodiment, the non endogenous ERRα transgene is a human transgene. In an especially preferred embodiment, the non endogenous transgene will be expressed in obesity-implicated cells and tissues.

Thus, the present invention relates to a knock-in approach, by which a wild type or mutant copy of the ERRα gene (i.e. human) is introduced or replaces the disrupted copy of the endogenous ERRα gene. The knock-in approach has been described (Hanks et al. (1995) Science 269:679-682) and has been shown to enable the expression of the non-endogenous copy of the gene in the same cells as that of the endogenous gene.

In a related aspect, the present invention relates to the use of such non- human transgenic mammals expressing a non-endogenous ERRα transgene to screen for a compound or agent that modulates ERRα orphan nuclear receptor activity, the method including: exposing the non-human transgenic mammal of the invention to the candidate compound, and determining the activity of the ERRα orphan nuclear receptor in the mammal, wherein an increase in the receptor activity as compared to untreated non-human mammals is indicative-of a compound being capable of increasing ERRα orphan nuclear receptor activity, while a decrease in the receptor activity as compared to untreated non-human mammals is indicative of a compound being capable of decreasing ERRα orphan nuclear receptor activity. In a preferred embodiment, the method further includes a determination of body or physiology parameters. Non-limiting examples thereof comprise a determination of: mass, body temperature, body fat content, fat to lean mass ratio, white adipose tissue deposits, basal metabolic rate, food intake, hepatic synthetic functions, fasting serum triglyceride, serum glucose levels, level of expression of uncoupling protein mRNA in brown adipose tissue (BAT) and skeletal muscle, adipocyte volume in fat pads, lipogenesis, and fatty acid esterification and oxydation.

As it will be understood by the person of ordinary skill, the present invention provides a number of significant advantages. For example, as for transgenic animals in general which have been shown to be useful for the investigation of biological processes and as animal model systems for general and specific aspects of health sciences in humans, the transgenic animals of the present invention provide a significant and pertinent model system for screening drugs to isolate therapeutic agents. In a particular embodiment, the novel transgenic animals of the present invention enable the selection and identification of modulators of the expression and/or activity of the ERRα orphan nuclear receptor. In a preferred embodiment, these agents have a use as anti-obesity, anti-fat deposition disorders, and/or anti-metabolic diseases associated with fat deposition disorders agents.

It will also be apparent to the person of ordinary skill, to which this application pertains, that the transgenic animals of the present invention can further be bred with other animals harboring known genotypes associated with fat deposition phenotypes and related disorders. Similarly the transgenic mammals of the present invention can be used in biochemical experiments and the like designed to further understand, dissect and/or treat obesity and related disorders.

It will also be apparent that the cells and tissues of the transgenic animals of the present invention can be useful in in vitro methods relating to fat deposition and related disorders (including rational design and/or screening of compounds which can modulate expression and/or activity of the ERRα orphan nuclear receptor. In a related aspect, the present invention further relates to cell lines in which the activity of ERRα has been inactivated or augmented. In addition to being derived from the transgenic animals of the present invention, such cell lines, can for example be derived as commonly known in the art using the construct of the present invention or derivatives or variants thereof. Such cell lines can be used similarly to the animals of the present invention to identify compounds which modulate ERRα level and/or acivity, dissect the physiological and biochemical function (including structure/function relationships, as they relate to fat deposition and the like) of ERRα. Thus, the present invention also relates to established cell lines or primary cells derived from an animal of the present invention. In one embodiment, fat pads from a transgenic mouse of the present invention was used to obtain primary cells which were grown and used in in vitro methods (i.e. insulin effect, gucose uptake, lipogenesis measurements and the like). Such experiments validated these cells as a pertinent tool for the methods and uses of the present invention.

Having determined that ERRα is involved in fat deposition and related disorders, as described herein, the present invention identifies ERRα as a target for therapy and diagnosis of fat deposition and related disorders. Further, the present invention provides the means to modulate the activity of ERRα. For example, antisense to ERRα can be used to decrease or abrogate the expression of ERRα polypeptide. This is expected to be associated with a lean phenotype. Antibodies, peptides, steroid-like compounds, pharmaceutical ligands, antagonists of ERRα receptor, and the like could be used with the same effect on the modulation of receptor ERRα activity. Alternatively, in certain embodiments, the fat deposition could be increased by for example overexpressing ERRα in cells or tissues. Of course, the non-limiting agents mentioned above could also act as stimulators or agonists of ERRα receptor activity.

Although the instant description focuses on mammalian transgenic animals, the present invention may also find utility in less common transgenic animals such as transgenic poultry. The production of leaner poultry might also be an advantage in the meat industry.

Having now identified ERRα as a target for fat tissue growth modulation, glucose metabolism, fat modulation, weight gain and the like, the present invention opens the way to the identification of further targets in the same pathway. Non-limiting examples of such targets include ERRβ, ERRγ, genes encoding enzymes involved in lipid metabolism whose expression is modulated by ERRα and related family members.

In accordance with the present invention, there is thus provided a non-human transgenic animal whose germ cells and somatic cells contain a knockout mutation in the endogenous ERRα orphan nuclear receptor gene, and wherein the transgenic animal shows a phenotype of an altered fat and/or glucose metabolism as compared to a control animal.

In accordance with the present invention, there is also provided a method of producing a non-human transgenic animal, in which at least some cells thereof contain an altered gene encoding an altered ERRα. The altered gene has been targeted to disrupt the endogenous ERRα gene in the transgenic animal. The method comprises:

a) providing an altered gene encoding the altered form of ERRα and designed to target and disrupt the endogenous ERRα gene of an embryonic stem cells (ES) of the animal;

b) introducing the altered gene in the ES cells;

c) selecting ES cells in which the altered ERRα gene has disrupted the endogenous ERRα gene;

d) injecting the selected ES cells of c) into blastocysts;

e) implanting the blastocysts of d) in a pseudopregnant animal; and

f) producing a non-human transgenic animal having at least some cells having the altered ERRα gene encoding the altered ERRα.

In addition, in accordance with the present invention, there is also provided a method of producing the non-human transgenic animal of the present invention. The method comprises:

(a) providing a non-human transgenic animal lacking detectable levels of ERRα orphan nuclear receptor gene and exhibiting a lean phenotype;

(b) introducing a non endogenous ERRα orphan nuclear receptor transgene encoding a functional ERRα orphan nuclear receptor gene into the pronucleus of a zygote derived from the animal of a), the zygote containing a homozygous disruption of the endogenous ERRα orphan nuclear receptor gene;

c) transplanting the animal zygote into a pseudopregnant compatible animal;

(d) allowing the zygote to develop to term;

(e) obtaining a founder animal carrying the transgene; and

(f) breeding the founder animal with a wild-type animal to obtain progeny that express the non endogenous ERRα orphan nuclear receptor transgene at levels sufficient to functionally complement the disrupted ERRα receptor activity.

Further, in accordance with the present invention, there is also provided a method for screening and identifying a compound which modulates ERRα orphan nuclear receptor activity. The method includes:

a) exposing the non-human transgenic animal in accordance with the present invention to a candidate compound, and;

b) determining the activity of the ERRα orphan nuclear receptor in the animal, where an increase in the receptor activity as compared to an unexposed non-human animal is indicative of a compound being capable of increasing ERRα orphan nuclear receptor activity, while a decrease in the receptor activity as compared to an unexposed non-human animal, is indicative of a compound being capable of decreasing ERRα orphan nuclear receptor activity.

Similarly, in accordance with the present invention, there is also provided a method of identifying an agent which modulates fat and/or glucose metabolism in vivo which comprises:

a) administering an agent suspected of being a modulator of ERRα activity and/or level in an animal;

b) measuring lipid and/or glucose levels in the animal of step a) and comparing same with that of a control animal not having been administered the agent, wherein a difference in lipid and/or glucose levels of the animal of step a) as compared to that of the control animal, identifies the agent as a modulator of fat and/or glucose metabolism in vivo.

As well, there is also provided a method of identifying an agent which modulates fat and/or glucose metabolism in vivo which comprises:

a) providing a promoter operably linked to a selectable or assayable marker, the promoter being modulated by ERRα;

b) measuring or selecting for the marker in a presence and in an absence of an agent suspected of modulating the promoter modulating activity of ERRα, thereby identifying an agent which modulates ERRα activity wherein a difference in the transcriptional activity in the presence of the agent, as compared to that in the absence thereof, identifies the agent as a modulator of ERRα activity;

c) administering the agent identified in b) to a non-human transgenic animal according to the present invention; and

d) measuring lipid and/or glucose levels in the animal of step c) and comparing same with that of a control animal, not having been administered the agent, wherein a difference in lipid and/or glucose levels of the animal of step c) as compared to that of the control animal identifies the agent as a modulator of fat and/or glucose metabolism in vivo.

Furthermore, in accordance with the present invention, there is provided a modulator of fat and/or glucose metabolism in vivo identified by a method of the present invention.

In accordance with the present invention, there is also provided a method of modulating fat tissue growth and/or weight gain. The method comprises administering to an animal an agent which modulates the promoter activity of a gene, wherein the promoter comprises cis-acting elements selected from the group consisting of:

i) an estrogen response element;

ii) TGA AGG TCA;

iii) AGG TCA NNN TGA CCT; and

iv) functional variants of i-iii)

such as to modulate the level of the gene, thereby modulating fat tissue growth and/or weight gain in the animal.

In accordance with another embodiment of the present invention, there is provided a method of determining whether an agent modulates fat tissue growth and/or weight gain in an animal comprising:

a) providing a transcriptionally active preparation of ERRα or related factors and a DNA sequence comprising a promoter having a cis-acting sequence which modulates activity thereof by an interaction thereto of said ERRα and related factors;

b) measuring the transcriptional activity of the promoter or of a binding of at least ERRα or related factors to the cis-acting sequence in a presence and in an absence of an agent suspected of modulating the transcriptional activity of the promoter or the binding of the factors to the cis-acting sequence, thereby identifying an agent which modulates transcription of the promoter and wherein a difference in the transcriptional activity and/or binding in the presence of the agent, as compared to that in the absence thereof identifies the agent as a modulator of transcription;

c) administering the agent identified in b) to a non-human transgenic animal according to one of claims 1 to 7; and

d) measuring fat tissue growth and/or weight gain in the animal of step c) and comparing same with that of a control animal, not having been administered the agent, wherein a difference in fat tissue growth and/or weight gain of the animal of step c) as compared to that of the control animal identifies the agent as a modulator of fat tissue growth and/or weight gain in vivo.

In accordance with yet another embodiment of the present invention, there is provided a method of treating and/or preventing obesity, comprising administering to an obese animal, or an animal susceptible of becoming obese, an agent which modulates the promoter activity of a promoter comprising a cis-acting element selected from the group consisting of:

i) an estrogen response element;

ii) TGA AGG TCA;

iii) AGG TCA NNN TGA CCT; and

iv) functional variants of i-iii)

wherein the cis-acting element is capable of binding to ERRα.

And yet in accordance with a further embodiment of the present invention, there is provided a method of determining whether an agent modulates obesity in an animal comprising:

a) providing a transcriptionally active preparation of ERRα or related factors and a DNA sequence comprising a promoter having a cis-acting sequence which modulates activity thereof by an interaction thereto of the ERRα and related factors;

b) measuring the transcriptional activity of the promoter or of a binding of at least ERRα or related factors to the cis-acting sequence in a presence and in an absence of an agent suspected of modulating the transcriptional activity of the promoter or the binding of the factors to the cis-acting sequence, thereby identifying an agent which modulates transcription of the promoter and wherein a difference in the transcriptional activity and/or binding in the presence of the agent, as compared to that in the absence thereof identifies the agent as a modulator of transcription;

c) administering the agent identified in b) to a non-human transgenic animal according to one of claims 1 to 7; and

d) assessing obesity in the animal of step c) and comparing same with that of a control animal, not having been administered the agent, wherein a difference in obesity of the animal of step c) as compared to that of the control animal identifies the agent as a modulator of obesity in vivo.

For the purpose of the present invention, the following abbreviations and terms are defined below.

Definitions

As used herein, the terminology “transgenic animal” refers to any animal which harbors a nucleic acid sequence having been inserted into a cell and having become part of the genome of the animal that develops from that cell. In a preferred embodiment, the transgenic animal is a mammal, in an especially preferred embodiment, the transgenic mammal is a mouse. However, other transgenic animals are encompassed as within scope of the present invention. Non-limiting examples of such transgenic animals include transgenic rodents (i.e. rats, hamsters, guinea pigs, and rabbits), and transgenic pigs, cattle and sheep, as well as transgenic poultry. Techniques for the preparation of such transgenic animals are well known in the art (e.g. introducing a transgene in ES cells; microinjecting the transgene into the male pronucleus of a fertilized egg; or infecting a cell with a recombinant virus). Indeed, lean transgenic animals may find utility in the food industry, in view of the increasing awareness of consumers to the degree of fat in meat products.

As used herein, “hon-human transgenic animal” is any non-human animal in which at least one cell comprises genetically altered information through known means such as microinjection, virus-delivered infection, or homologous recombination. In one particularly preferred embodiment of the present invention, the transgenic animal is a transgenic mouse, in which the genetic alteration has been introduced in a germ-line cell such, that it enables the transfer of this genetic alteration to the offsprings thereof. Such offsprings, containing this genetic alteration, are also transgenic mice.

The terminology “gene knockout” or “knockout” refers to a disruption of a nucleic acid sequence which significantly reduces and preferably suppresses or destroys the biological activity of the polypeptide encoded thereby. For example, ERRα knockout animal refers to an animal in which the expression of ERRα has been reduced or suppressed by the introduction of a recombinant nucleic acid molecule comprising ERRα sequences that disrupt at least a portion of the genomic DNA sequence encoding ERRα in the animal. A knockout animal might have one or both copies of the preselected nucleic acid sequence disrupted. In the latter case, in which a homozygous disruption is present, the mutation is termed a “null” mutation. In a case where only one copy of a preselected nucleic acid sequence is disrupted, the knockout animal is a “heterozygous knockout animal”.

The terminology “estrogen response elements” or “estrogen cis-acting elements” refers to well-known nucleic acid sequences to which transcription factors such as the orphan nuclear receptor ERRα can bind, thereby having the potential to modulate the promoter activity of a promoter comprising such response or cis-acting elements. These cis-acting elements or estrogen response elements also termed “ERE” or “IR3” are well-known in the art (Petterson, 1996, Mech. Dev. 54:211-223). In Petterson et al. (1996, supra), it is for example taught that the perfect inverted repeat (IR) of the estrogen response element to which ERRα can bind has sequence AGG TCA NNN TGA CCT. It is also known from Sladek et al., 1997, Bonnelye et al., 1997 and Johnston et al., 1997 that this acting element comprising the sequence TGA AGG TCA can also bind ERRα and related factors.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

The present description refers to a number of routinely used recombinant DNA (rDNA) technology terms. Nevertheless, definitions of selected examples of such rDNA terms are provided for clarity and consistency.

“nucleic acid molecule”, refers to a polymer of nucleotides. Non-limiting examples thereof include DNA (i.e. genomic DNA, cDNA) and RNA molecules (i.e. mRNA). The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (i.e. DNA or RNA) for practising the present invention may be obtained according to well known methods.

As used herein, the term “physiologically relevant” is meant to describe the functional relevance of a nucleic acid and/or protein in its natural setting.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 nucleotides, and they may be adapted to be especially suited to a chosen nucleic acid amplification system. As commonly known in the art, the oligonucleotide probes and primers can be designed by taking into consideration the melting point of hydrizidation thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “oligonucleotide” or “DNA” molecule or sequence refers to a molecule comprised of the deoxyribonucleotides adenine (A), guanine (G), thymine (T) and/or cytosine (C), in a double-stranded form, and comprises or includes a “regulatory element” according to the present invention, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction.

“Nucleic acid hybridization” refers generally to the hybridization of two single-stranded nucleic acid molecules having complementary base sequences, which under appropriate conditions will form a thermodynamically favoured double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labelled probe in a solution containing 50% formamide, high salt (5×SSC or 5×SSPE), 5×Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 1989, supra).

Probes of the invention can be utilized with naturally occurring sugar-phosphate backbones as well as modified backbones including phosphorothioates, dithionates, alkyl phosphonates and a-nucleotides and the like. Modified sugar-phosphate backbones are generally taught by Miller, 1988, Ann. Reports Med. Chem. 23:295 and Moran et al., 1987, Nucleic acid molecule. Acids Res., 14:5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labelled proteins could also be used to detect a particular nucleic acid sequence to which it binds. Other detection methods include kits containing probes on a dipstick setup and the like. Of course, it will be understand that the present invention lends itself to semi- or full-automated screening techniques. A non limiting of such a screening technique includes the known gene chips technology.

Although the present invention is not specifically dependent on the use of a label for the detection of a particular nucleic acid sequence, such a label might be beneficial, by increasing the sensitivity of the detection. Furthermore, it enables automation. Probes can be labelled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (i.e. uniformly labelled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthetised chemically or derived by cloning according to well known methods.

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise the a specific polypeptide or protein. It will be readily recognized by the person of ordinary skill, that the nucleic acid sequence of the present invention can be incorporated into anyone of numerous established kit formats which are well known in the art.

A “heterologous” (i.e. a heterologous gene) region of a DNA molecule is a subsegment segment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The terminology “endogenous gene” generally defines the gene which has been disrupted to produce the knockout transgenic animal. In a particular embodiment relating to a knockout mice, the endogenous ERRα gene is the mouse ERRα gene. In a related aspect, the terminology “non-endogenous transgene” should be generally understood as a transgene which is not in its natural setting (e.g. different expression control elements), was isolated from a different species (e.g. human), or has been engineered to display a new characteristic (e.g. an engineered mutant gene).

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), and the mRNA translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites. In addition, the expression control sequence can confer constitutive or inducible expression upon the sequence to which it is operably linked.

Prokaryotic expressions are useful for the preparation of large quantities of the protein encoded by the DNA sequence of interest. This protein can be purified according to standard protocols that take advantage of the intrinsic properties thereof, such as size and charge (i.e. SDS gel electrophoresis, gel filtration, centrifugation, ion exchange chromatography . . . ). In addition, the protein of interest can be purified via affinity chromatography using polyclonal or monoclonal antibodies. Polyclonal antibodies which can be used in the context of the present invention have been described (Sladek et al. Supra). The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

As used herein, the designation “functional derivative” denotes, in the context of a functional derivative of a sequence whether an nucleic acid or amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid as chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention.

The functional derivatives of the present invention can be synthesized chemically or produced through recombinant DNA technology. all these methods are well known in the art.

As used herein, “chemical derivatives” is meant to cover additional chemical moieties not normally part of the subject matter of the invention. Such moieties could affect the physico-chemical characteristic of the derivative (i.e. solubility, absorption, half life and the like, decrease of toxicity). Such moieties are exemplified in Remington's Pharmaceutical Sciences (e.g. 1980). Methods of coupling these chemical-physical moieties to a polypeptide are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in all other cellular components.

As used herein, the terms “molecule”, “compound”, “agent” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non limiting examples of molecules include nucleic acid molecules, peptides, antibodies, carbohydrates and pharmaceutical agents. The agents can be selected and screened by a variety of means including random screening, rational selection and by rational design using for example protein or ligand modelling methods such as computer modelling. The terms “rationally selected” or “rationally designed” are meant to define compounds which have been chosen based on the configuration of the interaction domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modelling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions in which the physiology or homeostasis of the cell and/or tissue is compromised by a defect in ERRα or in pathways converging thereon or therefrom. Alternatively, the molecules identified in accordance with the teachings of the present invention find utility in the development of more efficient agents which modulate ERRα level or activity. In a preferred embodiment, the molecules are agonists and antagonists compounds of ERRα activity. Such compounds can be steroid-like on non-steroidal compounds. Of course, these compounds can be identified from libraries (e.g. a combinatorial library). Since the ERRα receptor is phosphorylated in vivo, compounds which modulate ERRα receptor activity through phosphorylation could also be identified. The compounds identified in accordance with the present invention could be modified as known by the person of ordinary skill so as to target a chosen or specific tissue- or cell-type.

In one embodiment, agonists or antagonists of ERRα can be detected and selected by contacting the indicator cell or animal with a compound or mixture or library of molecules for a fixed period of time and an activity of ERRα is then determined.

In one particular embodiment, the level of gene expression of ERRα can be determined directly or indirectly (e.g. through the level of a reporter gene such as luciferase, or β-gal) within the treated cells or animal and compared to the level thereof in the absence of the molecules(s). The difference between the levels of gene expression indicates whether the molecule(s) agonizes or antagonizes the expression of ERRα. The magnitude of the level of the effect of the molecule(s) (treated vs. untreated cells) provides a relative indication of the strength of that molecule(s). The same type of approach can also be used in the presence of an antagonist(s).

As well, having identified ERRα as a target for lipogenesis, fatty acid esterification and fatty acid oxydation modulation, ERRα can be be used in a number of in vitro and in vivo assays to identify ligands therefor and dissect its structure/function relationship. Non limiting examples thereof include binding assays and the two hybrid system technology, as well known in the art (Ausubel et al., 1994, supra). This assay has proven beneficial to test compounds or a library thereof. Thus, the invention also covers ERRα-expressing cells (prokaryotes, lower and higher eukaryotes) or variants thereof to identify mutations which modulate ERRα activity or compunds which have ERRα modulating effects.

The present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility bu using nucleotide analogs and/or substituting chosen chemical fragments thereof, as commonly known in the art.

Of course, the cells or animals in accordance with the present invention can be used to identify antagonists.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. Of course, an advantage might be rendered moot if two polypeptides or interacting domains thereof are tested. It will be understood that extracts from mammalian cells for example could be used in certain embodiments, to compensate for the lack of certain factors in a chosen indicator cell.

An indicator cell in accordance with the present invention can be used to identify antagonists. For example, the test molecule or molecules are incubated with the host cell in conjunction with one or more agonists held at a fixed concentration. An indication and relative strength of the antagonistic properties of the molecule(s) can be provided by comparing the level of gene expression in the indicator cell in the presence of the agonist, in the absence of test molecules versus in the presence thereof. Of course, the antagonistic effect of a molecule could also be determined in the absence of agonist, simply by comparing the level of expression of the reporter gene product in the presence and absence of the test molecule(s).

It shall be understood that the “in vivo” experimental model can also be used to carry out an “in vitro” assay. For example, cellular extracts from the indicator cells and/or cellular extracts from the non-human transgenic animals of the present invention can be prepared and used in one of the in vitro method of the present invention or an in vitro method known in the art. Non-limiting examples of such assays are taught in U.S. Pat. No. 5,298,429. It should be noted that U.S. Pat. No. 5,298,429 also teaches the sequence of ERRα and ERRβ from human, as well as the significant conservation in the sequence of ERRα, ERRβ, and related family members.

In one particular embodiment, an “indicator cell” can be designed so as to express ERRα so as to modulate a promoter operably linked to a reporter gene, or to an identifiable or selectable phenotype or characteristic such that it provides an assessment of the activity and/or level of ERRα. Such indicator cells can be used in the screening assays of the present invention. In certain embodiments, the indicator cells have been engineered so as to express a chosen derivative, fragment, homolog, or mutant of ERRα. The cells can be prokaryotic cells, yeast cells or higher eukaryotic cells such as mammalian cells (WO 96/41169). In one particular embodiment, the indicator cell is a yeast cell harboring vectors enabling the use of the two hybrid system technology, as well known in the art (Ausubel et al., 1994, supra) and can be used to test a compound or a library thereof. In one embodiment, a reporter gene encoding a selectable marker or an assayable protein can be operably linked to a control element such that expression of the selectable marker or assayable protein is dependent on the interaction of ERRα with an interacting protein thereof. Such an indicator cell could be used to rapidly screen at high-throughput a vast array of test molecules. In a particular embodiment, the reporter gene is luciferase or β-Gal.

In one embodiment, at least one of the ERRα and a protein or domain thereof with which it interacts may be provided as a fusion protein. The design of constructs therefor and the expression and production of fusion proteins are well known in the art (Sambrook et al., 1989, supra; and Ausubel et al., 1994, supra). In a particular embodiment, both interaction domains are part of fusion proteins.

Non limiting examples of such fusion proteins include a hemaglutinin fusions, Gluthione-S-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

In certain embodiments, it might also be beneficial to fuse the interaction domains of the present invention to signal peptide sequences enabling a secretion of the fusion protein from the host cell. Signal peptides from diverse organisms are well known in the art. Bacterial OmpA and yeast Suc2 are two non limiting examples of proteins containing signal sequences. In certain embodiments, it might also be beneficial to introduce a linker (commonly known) between the interaction domain and the heterologous polypeptide portion. Such fusion protein find utility in the assays of the present invention as well as for purification purposes, detection purposes and the like.

For certainty, the sequences and polypeptides useful to practice the invention include without being limited thereto mutants, homologs, subtypes, alleles and the like. It shall be understood that generally, the sequences of the present invention should encode a functional (albeit defective) interaction domain. It will be clear to the person of ordinary skill that whether an interaction domain of the present invention, variant, derivative, or fragment thereof retains its function in binding to its partner or in modulating transcription can be readily determined by using the teachings and assays of the present invention and the general teachings of the art.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents. Further, the DNA segments or proteins according to the present invention can be introduced into individuals in a number of ways. For example, a chosen cell type cell can be isolated from the afflicted individual, transformed with a DNA construct according to the invention and reintroduced to the afflicted individual in a number of ways, including intravenous injection. Alternatively, the DNA construct can be administered directly to the afflicted individual, for example, by injection in the bone marrow. The DNA construct can also be delivered through a vehicle such as a liposome, which can be designed to be targeted to a specific cell type, and engineered to be administered through different routes.

In one particular embodiment, the present invention provides the means to treat weight-related diseases or conditions comprising a decrease or total eradication of ERRα expression. It will be recognized that having shown that the absence of ERRα expression reduces fat tissue, provides numerous means of achieving fat reduction in animals.

For administration to humans, the prescribing medical professional will ultimately determine the appropriate form and dosage for a given patient, and this can be expected to vary according to the chosen therapeutic regimen (i.e. DNA construct, protein, cells), the response and condition of the patient as well as the severity of the disease.

Composition within the scope of the present invention should contain the active agent (i.e. fusion protein, nucleic acid, and molecule) in an amount effective to achieve the desired therapeutic effect while avoiding adverse side effects. Typically, the nucleic acids in accordance with the present invention can be administered to mammals (i.e. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.1 ng to 1 g/kg/day, and preferably 10 mg to 50 mg/kg/day will be administered to the mammal.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the targeted disruption of the Estrra gene and heterozygote inbreeding analysis. a, Structure of the ERRα locus, targeting vector, and recombinant allele. Top, map of the wild type locus: exons are indicated by black boxes. E2 encodes the upstream zinc-binding motif of the ERRα DNA-binding domain. Center, targeting construct. Bottom, map of the targeted allele, showing replacement of exon 2 sequences by the neo^{r} cassette. The restriction enzyme digests and the probes used to characterize the knockout mice are illustrated. B, BamHI; H, HindIII. b, Southern blot analysis of targeted ES clones. DNA from parental ES cells (R1) and two targeted clones (57 and 62) was digested with BamHI and hybridized to the 3′ probe. The positions of bands corresponding to the wild-type (10.7 kb) and targeted alleles (4.5 kb) are indicated (upper panel). Single integration of the targeting construct in targeted ES cell clones was confirmed with a neo^{r} probe: a single hybridizing band (6.0 kb) is present in the targeted lines (lower panel). c, Southern blot analysis of genotypes of 28 d old pups from a heterozygote intercross: the litter contains viable homozygous null mice. d, Northern blot analysis of RNA obtained from the kidneys of the progeny of heterozygous intercrosses. ERRα expression is not detected in RNA samples obtained from homozygous null mutants.

FIG. 2 shows the phenotypic analysis of Estrra null mutants. a, Mutant animals display decreased weight gain. Growth curves were performed by weighing animals at the indicated ages: both male and female knockout mice display significantly reduced body weight in comparison to their wild-type littermates. Arrows indicate start of pre-pubertal growth spurt. b, Body composition of Estrra null mice shows decreased ratio of fat to lean mass. c, Estrra^{-/-} mice contain decreased body fat. Superficial carcass dissection of two 20 week old male mice shows the decreased body fat content of a 32.9 g knockout mouse (right) in comparison with his 38.1 g wild-type littermate (left). d, The difference in body composition is reflected by the relative sizes of the dissected fat pads.

FIG. 3 shows the analysis of intestinal lipid transport in Estrra null mutants. a, Thin layer chromatographic analysis of tissue lipid content. The intestines of Estrra^{-/-} mice contain decreased triglyceride and increased free fatty acids in comparison with their wild-type and heterozygous littermates. b, Analysis of glycerolipid synthesis in Estrra null mutants. Estrra−/− mice demonstrate reduced triglyceride synthesis in intestinal and hepatic whole cell extracts. c, Fat absorption profile. Estrra^{-/-} mice and littermate controls display similar rates of absorption of radiolabeled oleic acid.

FIG. 4 shows the analysis of adipocyte function in Estrra null mutants. a, Histologic studies of epididymal fat pads show that Estrra^{-/-} mice (lower panel) have decreased adipocyte volume in comparison to wild-type animals (upper panel). b, Estrra^{-/-} mice demonstrate decreased lipogenesis in comparison to littermate controls.

Following intraperitoneal injection with ³H₂O, Estrra mice incorporate 30-55% less ³H into adipose tissue and 50% less ³H into hepatic lipids. IF, inguinal fat; EF, epididymal fat; PF, perirenal fat.

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

The method of production and the transgenic animals of the present invention are described herein below. In general, these animals are produced by engineering a nucleic acid construct which can disrupt the expression of the endogenous ERRα gene (i.e., the murine ERRα gene). Using known methods, this construct is amplified in bacterial cells, purified, and transferred into ES cells or isolated oocytes. The transfected ES cells can then be injected into blastocysts to generate chimeras. The chimeras which transmit the mutation to their offspring are identified and selected. These animals can then be used as founder animals to obtain different animal lines, derived from breeding with chosen animals. Heterozygous animals can then be produced and further mated to generate a hybrid F1 cross. Further matings of the F1 heterozygotes produce the wild type, heterozygous and homozygous null mutants of ERRα (having both copies of the ERRα gene disrupted). The homozygous animals can then serve in a number of experiments. Non-limiting examples thereof include: the characterization of their phenotype, and a reconstitution of the ERRα activity by complementation by a non-endogenous copy of a wild type ERRα gene or mutant or variant ERRα gene. An animal (or cells derived therefrom) expressing a mutant form of ERRα gene (from human, for example) could be used to screen for compounds which modulate more specifically the mutant form of the ERRα gene.

The present invention therefore strongly indicates that ERRα is a direct regulator of fundamental cellular function. It is thus expected that this cellular function should occur accross species. The presence of the ERRα gene and its conservation among species (human, mice, rats, fish and lower organisms; Escriva et al. (1997) Proc. Natl. Acad. Sci. USA 94:6803-6808), support its essential role in physiology. Thus, the antagonists identified by the methods and assays of the present invention should find a utility in the treatment of obesity and other metabolic diseases associated with ERRα malfunction in humans.

The present invention is illustrated in further detail by the following non-limitin examples.

### EXAMPLE 1

Creation of Estrra^{-/-} Mice

Three overlapping λ clones containing the mouse Estrra locus were isolated from a 129Sv genomic library (gift of Dr. A. Joyner, Skirball Institute, New York) and characterized by restriction mapping and direct sequencing of the exon boundaries. The knockout construct was created using pNT (Tybulewicz et al., 1991) and contained 6.4 kb of genomic DNA flanking the second exon of Estrra. An endfilled 4.2 Kb BamHI/NotI fragment, lying upstream of the second exon, was cloned into the XhoI site of pNT, while a 2.2 Kb HindIII fragment was cloned between the neor and TK cassettes to provide the 3′ arm of the construct. Correct targeting of the Estrra locus replaces the receptor's second exon, which encodes a critical part of its DNA binding domain, with a neo cassette. The linearized construct was electroporated into R1 ES cells (Nagy and Rossant, 1993) which were selected with G418 (150 μg/mL) and gancyclovir (2 μM). Two ES cell clones were isolated and injected into C57BL6 blastocysts to generate chimeras, and three chimeras transmitted the mutation to their offspring. Heterozygous mice, generated by mating the chimeric animals with 129SvJ mice were mated with C57BL6 animals to generate hybrid F1 animals: physiologic studies were performed using the F2 null mutant and wild-type offspring obtained by mating the F1 hybrid heterozygotes. Complete disruption of the Estrra allele was verified by performing Northern blots using RNA obtained from placenta and kidneys of homozygous mutants.

### EXAMPLE 2

Physiological Parameters of Estrra^{-/-} Mice

Mice were housed in an SPF facility with a daily 12 h light cycle (7:00 to 19:00 h) and with free access to food and water. Between two and four mice were contained in each cage. Growth curves were obtained by weighing mice of defined ages between 10:00 and 12:00 h. Fasting serum and biochemical studies were performed between 10:00 to 12:00 h using animals that had been deprived of food for 18 hrs. Body composition was determined by desiccating mouse carcasses from which the intestines had been removed. Following desiccation, the carcass was homogenized and a 1 g aliquot was saponified using potassium hydroxide and extracted with petroleum ether. Following complete evaporation of the ether, the residue was weighed to determine fat content. Rectal temperature was measured using a rectal probe in animals housed at 29° C. and 4° C. Baseline biochemical studies were performed using serum samples obtained from tail bleeds of restrained animals at between 20 and 28 weeks of age. Enzymatic assays were used to determine serum triglycerides (GPO-PAP, Boehringer-Mannheim) and glycerol (TC Glycerin, Boehringer-Mannheim), glucose (Glucose Oxidase-Trinder, Sigma), free fatty acids (GPO-PAP Half Micro Test, Boehringer-Mannheim), and, β-hydroxybutyrate (TC β-hydroxybutyrate, Boehringer-Mannheim).

### EXAMPLE 3

Organ Lipid Content and Esterification Rates of Estrra^{-/-} Mice

Mice were allowed free access to food and water overnight. Experiments were performed between 09:00 and 11:00 h, at which time the animals were sacrificed by cervical dislocation and their tissues harvested and frozen in liquid nitrogen. To study tissue lipid content, the frozen tissues were pulverized on a precooled anvil and homogenized in cold 1×PBS. The homogenate was extracted using a 4:1 volume ratio of Folch buffer (chloroform: methanol). The extracted lipids were separated by thin-layer chromatography using a silica plate (Whatman LK5D) and visualized by iodine staining. Intestinal fatty acid esterification was studied using pulverized tissue, which was homogenized briefly in 1×PBS. Following brief centrifugation (13,000 rpm, 5 min, 4° C.) to pellet cell nuclei and membrane debris, the soluble protein fraction was extracted and quantified using the Bradford reagent. Between 50 and 200 micrograms of crude protein extract was used to study the incorporation of {9,10-3H}oleic acid (New England Nuclear) into glycerolipids using to previously published methods (Yasruel et al., 1991).

### EXAMPLE 4

Measurement of a Physiology Parameter of the Transgenic Mouse: Lipogenesis Rate Measurements of Estrra^{-/-} Mice

Mice were studied at 10:00 h following free access to food overnight. The animals were conditioned by sham intraperitoneal injections of water. On the day of the experiment, the animals were injected intraperitoneally with ³H₂O (0.5 mCi per 100 g body weight) and sacrificed by cervical dislocation 30 minutes later. Serum, adipose tissue and liver samples were harvested and stored at −80° C. The tissues were homogenized and heated in ethanolic KOH: the resulting extract, which contained saponified lipids, was acidified using concentrated sulfuric acid and extracted using petroleum ether. The extract was dried by evaporation and ³H incorporation determined by scintillation counting.

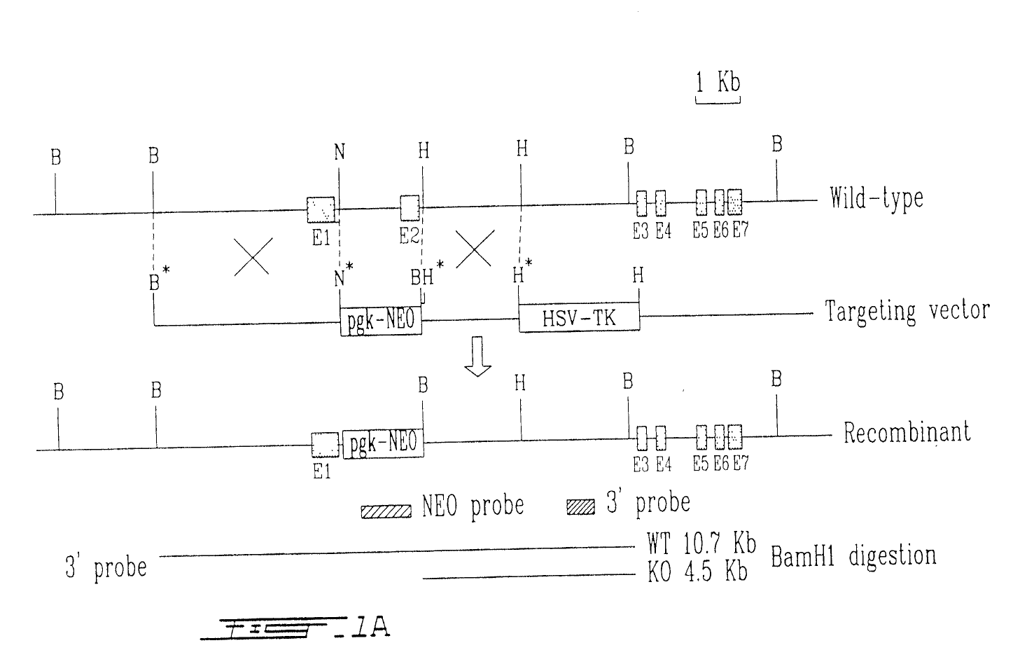

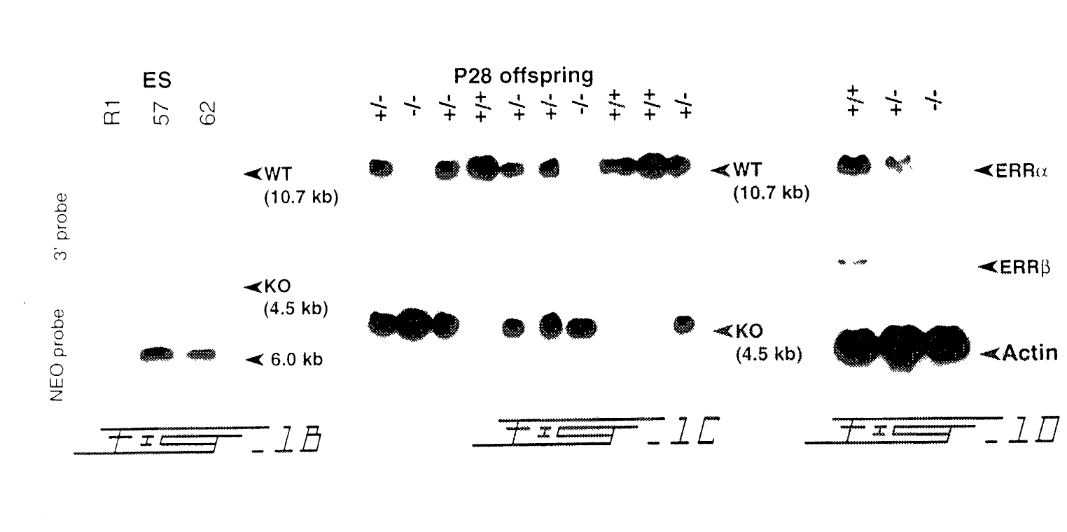

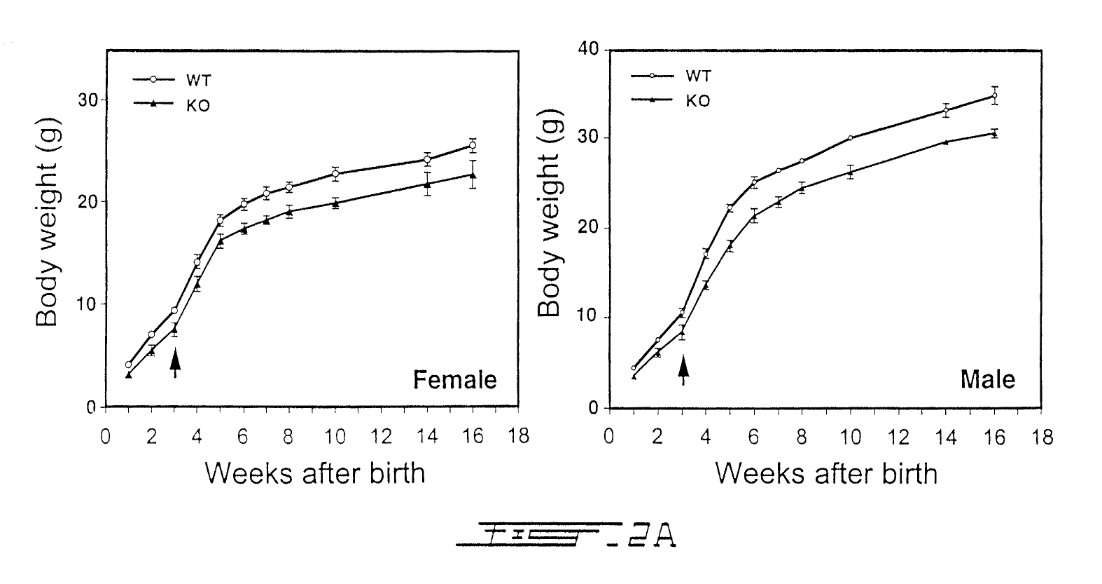

The Estrra gene was inactivated in embryonic stem (ES) cells using a targeting vector which replaces exon 2 of the receptor with the neor gene: this exon encodes a critical portion of the receptor's DNA binding domain (FIG. 1A). Two correctly targeted ES cell clone were obtained (FIG. 1B), one of which (clone #62) was injected into C57BL/6 blastocysts to generate chimeric animals. Three chimera transmitted the targeted allele to their offspring. Heterozygous mice were generated by mating the founder animals with 129/SvJ mice which were then mated with C57BL/6 animals to generate an hybrid F1 cross. Litters obtained from mating the F1 heterozygotes contained appropriate numbers of wild type, heterozygous and homozygous null animals (FIG. 1C). In addition, ERRα null mutants underwent grossly normal intrauterine development, were fertile, appeared healthy and did not exhibit increased mortality when compared to their wild-type littermates. Northern blot analysis of RNA obtained from the kidneys of homozygous mutants confirmed complete disruption of the Estrra locus: no ERRα transcripts were detected in tissues obtained from homozygous null mutants (FIG. 1D).

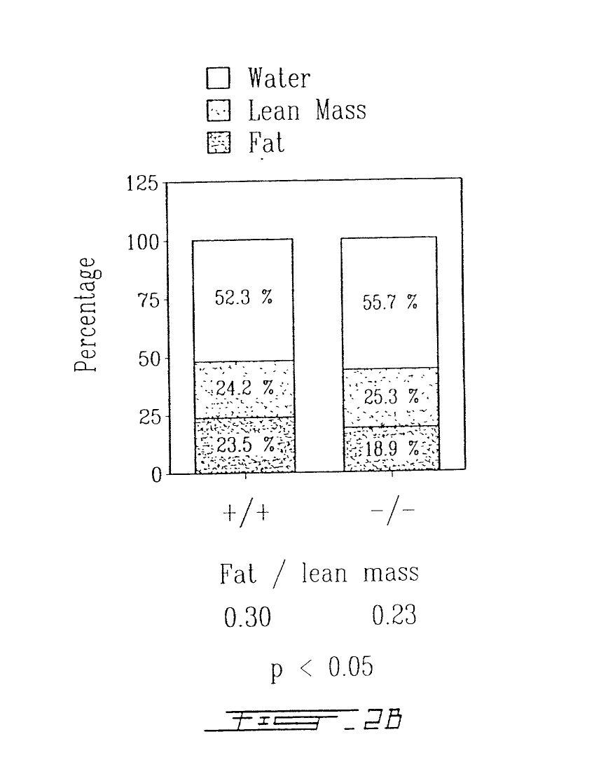

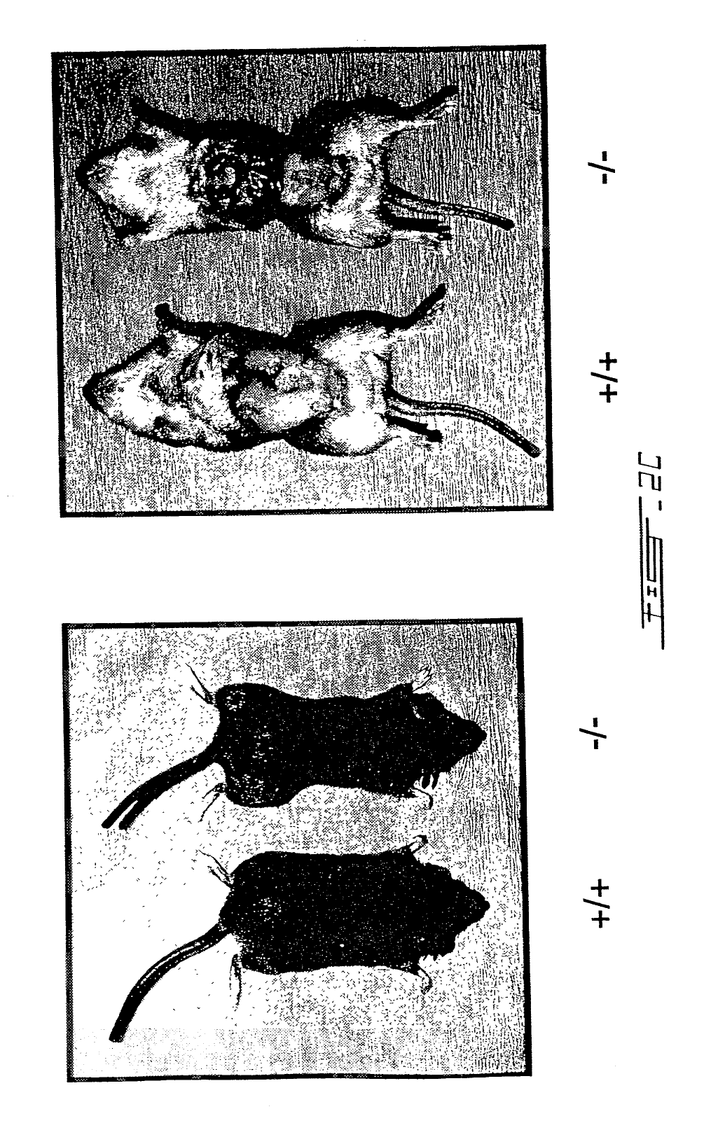

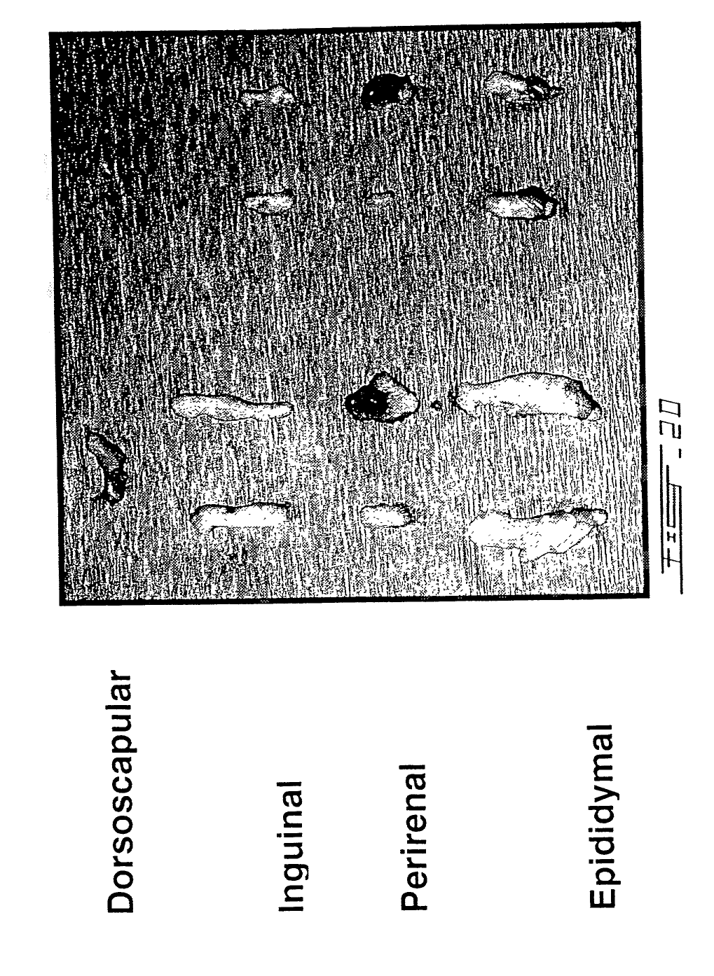

Phenotypic analysis of embryonic and post-natal mice was performed using F2 hybrid strain animals. Male and female Estrra^{-/-} mutants displayed significantly decreased body mass, which was not associated with changes in body length or in the time of onset of pre-pubertal growth (Table 1 and FIG. 2A). Body composition studies were performed using male animals, and revealed that ERRα null mutants contained 32% less body fat and a decrease in fat to lean mass ratio (Table 1 and FIG. 2B), and decreased white adipose tissue (WAT) deposits (FIG. 2C and D). Decreased food intake or increased fat excretion (Table 1) could not account for this alteration in body composition. In addition, fasting serum triglyceride and serum glucose levels were identical in wild-type and knockout animals, demonstrating that the mutant animals had normal hepatic synthetic function (Table 2).

TABLE 1
Physiological characterization of male ERRα mice

| Genotype | BW | Length (cm) | Fat (g) | Lean mass (g) | Water (g) | Food Intake (g/d) | Fecal Fat (%) |
|---|---|---|---|---|---|---|---|
| Control | 32.7 ± 1.0 | 9.6 ± 0.1 | 6.89 ± 0.70 | 6.86 ± 0.15 | 14.8 ± 0.22 | 5.17 ± 0.21 | 3.03 ± 0.22 |
| (+/+, +/−) | | | | | | | |
| Mutant | 27.3 ± 1.0 | 9.3 ± 0.4 | 4.64 ± 0.53 | 6.03 ± 0.18 | 13.1 ± 0.41 | 4.93 ± 0.27 | 3.03 ± 0.08 |
| (−/−) | | | | | | | |
| % of | 83.4% | 96.8% | 67.3% | 88.0% | 88.6% | 95.3% | 100.0% |
| control | | | | | | | |
| | p < 0.001 | NS | p < 0.02 | p < 0.01 | p < 0.01 | NS | NS |

TABLE 2
Biochemical characterization of male ERRα mice

| | Fasting | Fasting | Fasting |
|---|---|---|---|
| Genotype | BG (mg/dL) | TG (mg/dL) | FFA (μm) |
| Control | 111.0 ± 8.2 | 108.8 ± 8.7 | 596 ± 41 |
| (+/+) | | | |
| Mutant | 107.0 ± 7.6 | 107.2 ± 4.9 | 751 ± 72 |
| (−/−) | | | |
| % of control | 96.3% | 98.5% | 126% |
| | NS | NS | NS |

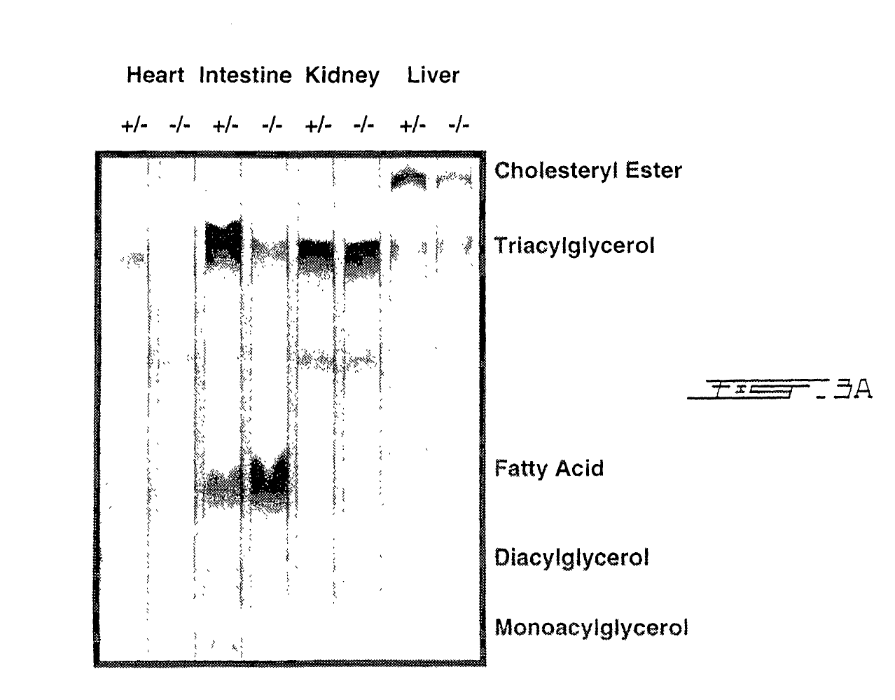

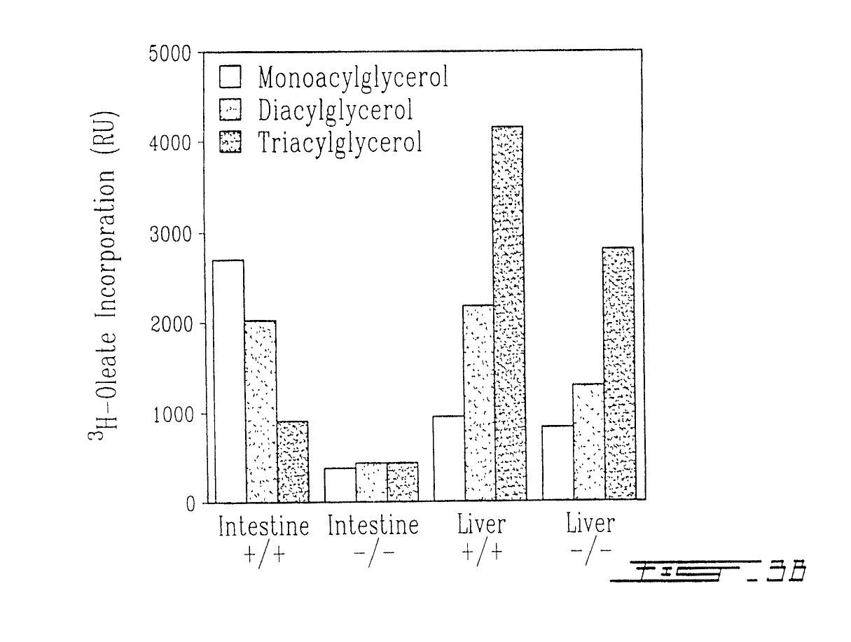

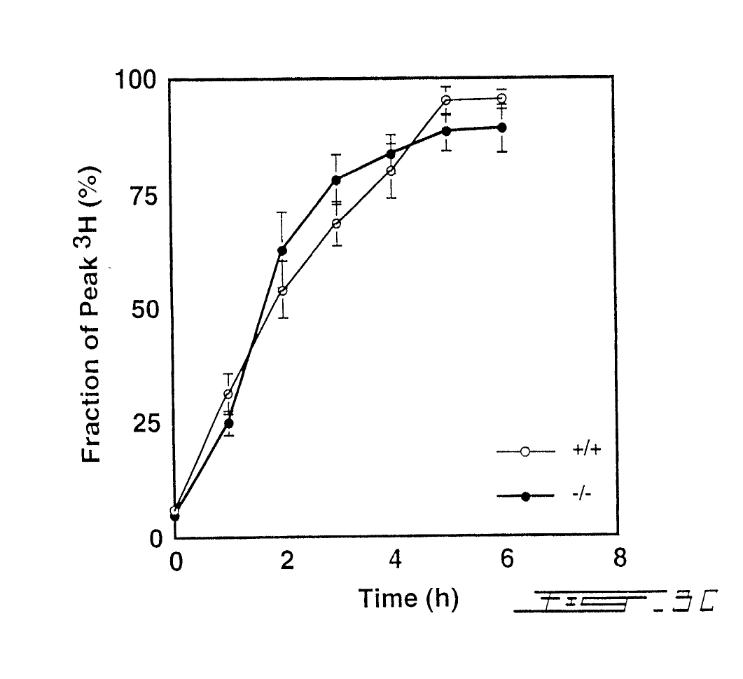

In order to further characterize the mechanism causing decreased fat mass in ERRα knockout mice, a determination as to whether these animals had subtle defects in intestinal triglyceride absorption was sought. TLC analysis of lipids obtained from whole cell extracts showed that the intestines of ERRα null mice contained increased levels of free fatty acids and decreased levels of triglycerides in comparison with wild type mice (FIG. 3A). This observation suggests that ERRα mice have decreased intestinal capacity for fatty acid esterification, a hypothesis that is confirmed by in vitro measurement of glycerolipid synthesis in intestinal whole cell extracts (FIG. 3B). Decreased intestinal fatty acid activation or esterification capacity would be expected to delay the rate at which dietary fatty acids and triglycerides are transferred across the intestine or to reduce the maximum serum triglyceride levels observed following a fat load; however, assessment of intestinal oleic acid transport in vivo shows that ERRα knockout animals and wild-type mice have similar rates of fatty acid absorption (FIG. 3C). Whether the abnormalities observed in intestinal lipid metabolism in vitro play major roles in the abnormal body composition observed in ERRα knockout mice is unclear; however, reduction of the maximal rate at which the intestines esterify dietary lipids may prevent Estrra^{-/-} mice from increasing intestinal energy transfer in order to compensate for other defects in fat or energy metabolism.

Previous studies have demonstrated that ERRα is highly expressed in brown adipose tissue (BAT) during murine development and that the receptor is upregulated during white and brown adipocyte differentiation in vitro (Sladek et al., 1997; Vega and Kelly, 1997). In addition, ERRα has been shown to modulate the activity of the medium chain acyl-coA dehydrogenase (MCAD) promoter, a key regulatory step in the fatty acid β-oxidation pathway (Sladek et al., 1997; Vega and Kelly, 1997). As dysregulation of BAT function has been associated with abnormalities of body composition, therefore, a characterization of BAT function in the ERRα knockout mice was carried out. ERRα null mutants had normal core body temperature and basal metabolic rate and displayed normal expression levels of uncoupling protein (UCP) mRNA in BAT (UCP-1) and skeletal muscle (UCP-2) (data not shown). Defects in fatty acid oxidation are frequently only apparent following situations of physiologic stress or food deprivation: neither prolonged cold exposure or fasts of up to 48 hour's duration resulted in any morbidity or mortality of Estrra−/− mice (data not shown). Taken together, these data suggest that the abnormal body composition seen in ERRα null mutants was not a result of increased thermogenesis or increased basal energy expenditure, and that the animals did not have physiologically significant defects in fatty acid β-oxidation.

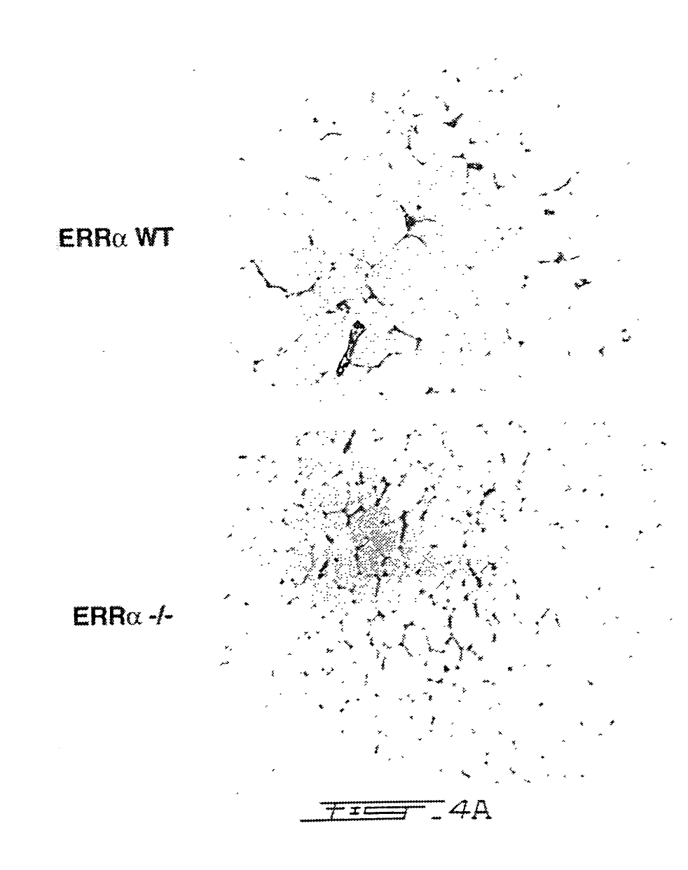

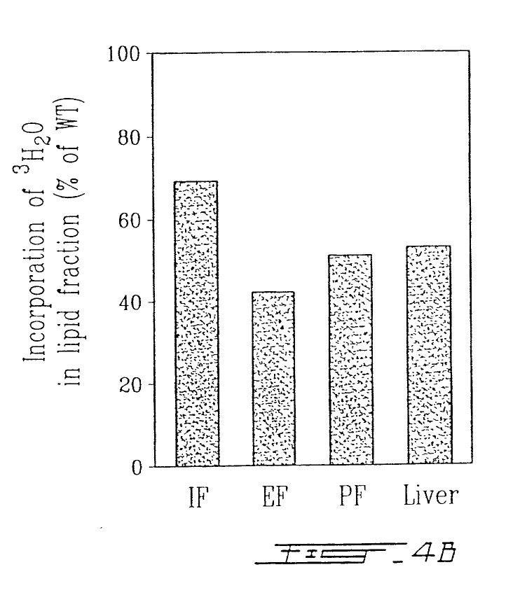

Fat pads obtained from Estrra mutants displayed decreased adipocyte volume in comparison to wild-type animals (FIG. 4A), which suggests that the decreased adipose tissue mass observed in Estrra−/− mice results from an imbalance between fatty acid synthesis and lipolysis rather than defects in adipocyte proliferation and differentiation. As ERRα expression is induced during early adipocyte differentiation in vitro (Sladek et al., 1997), it is possible that ERRα acts as a regulator of processes important for adipocyte function, such as fatty acid synthesis or esterification. In animals fed a standard laboratory diet, murine adipose tissue contains triglyceride formed from fatty acids that are synthesized de novo rather than from dietary lipid. Lipogenesis was assessed by treating Estrra^{-/-} mice with ³H₂O: the amount of radioactive label incorporated into triacylglycerol can be measured by saponification and ether extraction of adipose tissues and other organs. Estrra null mutants demonstrate significantly decreased lipogenesis in comparison to littermate controls: in particular, knockout animals show a 30-55% decrease in ³H incorporation into adipose tissue lipids and a 50% decrease in 3H incorporation into hepatic lipids (FIG. 4B). This observation demonstrates that adipose tissue of knockout mice possesses a defect in TG synthesis, which may result from decreased adipocyte and hepatic glycolysis activity, fatty acid synthesis or esterification.

Experiments performed using the Estrra−/− mice revealed that ERRα is a key regulator of fat metabolism, including intestinal fat transfer and esterification, as well as hepatic and adipocyte fat deposition. Estrra^{-/-} mice display decreased fat content associated with reduced intestinal fatty acid esterification rates and abnormal regulation of fat deposition and mobilization in adipocytes and liver. Previous in vitro studies have demonstrated that ERRα modulates the expression of MCAD, a key regulatory enzyme of fatty acid β-oxidation, a pathway which may also play a role in establishing the ERRα phenotype. The relative importance of each of these effects in establishing the body composition of ERRα mice remains to be determined. Since the Estrra^{-/-} mice show a normal level of energy intake, one would expect to observe an increase in energy expenditure to account for the decreased fat content of these mice. However, the sensitivity of fecal fat measurements and calorimetry experiments may not be sufficient to identify small differences between wild-type and knockout animals which over a period of time would be sufficient to explain the observed phenotype. Within these experimental limitations, the data presented herein demonstrate that ERRα mice are lean as a result of aberrant regulation of peripheral lipid mobilization. ERRα mice display an unique combination of properties that suggests that modulation of ERRα activity may provide an effective method to regulate fat metabolism and that ERRα would be a key drug target for the treatment of obesity and other disorders of fat deposition. In addition, the close linkage of ESTRRA and diabetes susceptibility locus IDDM4 (Sladek et al., 1997) together with physiological defects observed in Estrra^{-/-} mice suggests that drugs influencing ERRα activity could also be used to treat diabetes and other metabolic disorders.

Although the present invention has been described hereinabove by way of preferred embodiments thereof, it can be modified, without departing from the spirit and nature of the subject invention as defined in the appended claims.

References

Giguère, V., Yang, N., Segui, P., and Evans, R. M. (1988). Identification of a new class of steroid hormone receptors. Nature 331, 91-94.

Johnston, S. D., Liu, X., Zuo, F., Eisenbraun, T. L., Wiley, S. R., Kraus, R. J., and Mertz, J. E. (1997). Estrogen-related receptor α1 functionally binds as a monomer to extended half-site sequences including ones contained within estrogen-response elements. Mol. Endocrinol. 11, 342-352.

Nagy, A., and Rossant, J. (1993). Production of completely ES cell-derived fetuses. In Gene targeting: a practical approach., A. L. Joyner, ed. (Oxford: Oxford University Press), pp.147-169.

Petterson, 1996, Mech. Dev. 54:211-223.

Sladek, R., Bader, J.-A., and Giguère, V. (1997). The orphan nuclear receptor estrogen-related receptor α_is a transcriptional regulator of the human medium-chain acyl coenzyme A dehydrogenase gene. Mol. Cell. Biol. 17, 5400-5409.

Sladek, R., Beatty, B., Squire, J., Copeland, N. G., Gilbert, D. J., Jenkins, N. A., and Giguère, V. (1997). Chromosomal mapping of the human and murine orphan nuclear receptor ERRα (ESRRA) and ERRβ (ESRRB) and identification of a novel human ERRα-related pseudogene. Genomics, (in press).

Tybulewicz, V. L. J., Crawford, C. E., Jackson, P. K., Bronson, R. T., and Mulligan, R. C. (1991). Neonatal lethality and lymphopoenia in mice with a homozygous disruption of the c-abl proto-oncogene. Cell 65, 1153-1163.

Vanacker, J.-M., Bonnelye, E., Delmarre, C., and Laudet, V. (1998). Activation of the thyroid receptor α gene promoter by the orphan nuclear receptor ERRα. Oncogene 17, 2429-2435.

Vega, R. B., and Kelly, D. P. (1997). A role for estrogen-related receptor α in the control of mitochondrial fatty acid β-oxidation during brown adipocyte differentiation. J. Biol. Chem. 272, 31693-31699.

Yang, C., Zhou, D., and Chen, S. (1998). Modulation of aromatase expression in the breast tissue by ERRα-1 orphan receptor. Cancer Res. 58, 5695-5700.

Yang, N., Shigeta, H., Shi, H. P., and Teng, C. T. (1996). Estrogen-related receptor, hERR1, modulates estrogen receptor-mediated response of human lactoferrin gene promoter. J. Biol. Chem. 271, 5795-5804.

Yasruel, Z., Cianflone, K., Sniderman, A. D., Rosenbloom, M., Walsh, M., and Rodriguez, M. A. (1991). Effect of acylation stimulating protein on the triacylglycerol synthetic pathway of human adipose tissue. Lipids 26, 495-499.

What is claimed is:

1. A non-human transgenic animal whose germ cells and somatic cells contain a knockout mutation in the endogenous ERRα orphan nuclear receptor gene, and wherein said transgenic animal shows a phenotype of an altered fat and/or glucose metabolism as compared to a control animal.

2. The transgenic animal of claim 1, wherein said germ cells and somatic cells contain a homozygous disruption of said ERRα gene, and wherein said disruption comprises the insertion of a selectable marker sequence.

3. The non-human transgenic animal of claim 1 or 2, wherein said animal is a mammal.

4. The non-human transgenic animal of claim 3, wherein said animal is a mouse.

5. The non-human transgenic animal of claims 1 to 4, displaying a lean phenotype.

6. The non-human transgenic animal of one of claims 1 to 5, whose germ cells and somatic cells additionally comprise a transgene encoding a non endogenous ERRα orphan nuclear receptor gene, wherein said transgene is expressed at levels sufficient to complement the disrupted endogenous ERRα orphan nuclear receptor activity.

7. The non-human transgenic animal of claim 6, wherein said non endogenous ERRα orphan nuclear receptor gene is a human ERRα orphan nuclear receptor gene.

8. The non-human transgenic animal of claim 7, wherein said animal is a mouse and said non-endogenous ERRα is a human ERRα gene.

9. A cell line derived from the non-human transgenic animal of one of claims 1 to 8.

10. A method of producing a non-human transgenic animal, in which at least some cells thereof contain an altered gene encoding an altered ERRα, said altered gene having been targeted to disrupt the endogenous ERRα gene in said transgenic animal, said method comprising:
a) providing an altered gene encoding the altered form of ERRα and designed to target and disrupt said endogenous ERRα gene of an embryonic stem cells (ES) of said animal;
b) introducing said altered gene in said ES cells;
c) selecting ES cells in which said altered ERRα gene has disrupted said endogenous ERRα gene;
d) injecting said selected ES cells of c) into blastocysts;
e) implanting said blastocysts of d) in a pseudopregnant animal; and
f) producing a non-human transgenic animal having at least some cells having said altered ERRα gene encoding said altered ERRα.

11. The method of claim 10, wherein said non-human transgenic animal is a mouse.

12. A method of producing the non-human transgenic animal of claim 5, said method comprising:
(a) providing a non-human transgenic animal lacking detectable levels of ERRα orphan nuclear receptor gene and exhibiting a lean phenotype;
(b) introducing a non endogenous ERRα orphan nuclear receptor transgene encoding a functional ERRα orphan nuclear receptor gene into the pronucleus of a zygote derived from said animal of a), said zygote containing a homozygous disruption of the endogenous ERRα orphan nuclear receptor gene;
(c) transplanting said animal zygote into a pseudopregnant compatible animal;
(d) allowing said zygote to develop to term;
(e) obtaining a founder animal carrying said transgene; and
(f) breeding said founder animal with a wild-type animal to obtain progeny that express said non endogenous ERRα orphan nuclear receptor transgene at levels sufficient to functionally complement the disrupted ERRα receptor activity.

13. The method of claim 12, wherein said non-human transgenic animal is a mammal.

14. The method of claim 12, wherein said mammal is a mouse, and wherein said non-endogenous ERRα transgene is a human ERRα gene.

15. A method for screening and identifying a compound which modulates ERRα orphan nuclear receptor activity, the method including:
a) exposing the non-human transgenic animal of one of claims 5 to 7 to a candidate compound, and;
b) determining the activity of said ERRα orphan nuclear receptor in said animal, wherein an increase in the receptor activity as compared to an unexposed non-human animal is indicative of a compound being capable of increasing ERRα orphan nuclear receptor activity, while a decrease in said receptor activity as compared to an unexposed non-human animal, is indicative of a compound being capable of decreasing ERRα orphan nuclear receptor activity.

16. The method of claim 15, further comprising a determination of at least one parameter selected from the group consisting of: mass, body temperature, body fat content, fat to lean mass ratio, white adipose tissue deposits, basal metabolic rate, food intake, hepatic synthetic functions, fasting serum triglyceride, serum glucose levels, level of expression of uncoupling protein mRNA in brown adipose tissue (BAT) and skeletal muscle, adipocyte volume in fat pads, lipogenesis, fatty acid esterification and fatty acid oxydation.

17. A method of identifying an agent which modulates fat and/or glucose metabolism in vivo comprising:
a) administering an agent suspected of being a modulator of ERRα activity and/or level in an animal;
b) measuring lipid and/or glucose levels in the animal of step a) and comparing same with that of a control animal not having been administered said agent, wherein a difference in lipid and/or glucose levels of the animal of step a) as compared to that of the control animal, identifies said agent as a modulator of fat and/or glucose metabolism in vivo.

18. Method of identifying an agent which modulates fat and/or glucose metabolism in vivo comprising:
a) providing a promoter operably linked to a selectable or assayable marker, said promoter being modulated by ERRα;
b) measuring or selecting for said marker in a presence and in an absence of an agent suspected of modulating the promoter modulating activity of ERRα, thereby identifying an agent which modulates ERRα activity wherein a difference in the transcriptional activity in the presence of said agent, as compared to that in the absence thereof, identifies said agent as a modulator of ERRα activity;
c) administering said agent identified in b) to a non-human transgenic animal according to one of claims 1 to 7; and
d) measuring lipid and/or glucose levels in said animal of step c) and comparing same with that of a control animal, not having been administered said agent, wherein a difference in lipid and/or glucose levels of the animal of step c) as compared to that of said control animal identifies said agent as a modulator of fat and/or glucose metabolism in vivo.

19. The method of claim 18, where the agent is obtained from a library of compounds.

20. The method of claim 19, wherein the animal is a mammal.

21. The method of claim 20, wherein said mammal is a mouse or human.

22. A modulator of fat and/or glucose metabolism in vivo identified by any one of the methods of claims 18, 19, 20 or 21.

23. A method of modulating fat tissue growth and/or weight gain, comprising:
a) administering to an animal an agent which modulates the promoter activity of a gene, wherein said promoter comprises cis-acting elements selected from the group consisting of:
i) an estrogen response element;
ii) TGA AGG TCA;
iii) AGG TCA NNN TGA CCT; and
iv) functional variants of i-iii)
such as to modulate the level of said gene, thereby modulating fat tissue growth and/or weight gain in said animal.

24. The method of claim 23, wherein said agent modulates said promoter activity of said gene, by decreasing a level and/or activity of ERRα.

25. The method of claim 24, wherein said agent is an antibody specific to ERRα, or an epitope-bearing portion thereof.

26. The method of claim 23, wherein said modulation of said promoter activity is effected by inhibition of ERRα synthesis.

27. The method of claim 26, wherein said agent comprises an antisense RNA, complementary to a nucleotide sequence encoding ERRα.

28. A method of determining whether an agent modulates fat tissue growth and/or weight gain in an animal comprising:
a) providing a transcriptionally active preparation of ERRα or related factors and a DNA sequence comprising a promoter having a cis-acting sequence which modulates activity thereof by an interaction thereto of said ERRα and related factors;
b) measuring said transcriptional activity of said promoter or of a binding of at least ERRα or related factors to said cis-acting sequence in a presence and in an absence of an agent suspected of modulating the transcriptional activity of said promoter or the binding of said factors to said cis-acting sequence, thereby identifying an agent which modulates transcription of said promoter and wherein a difference in the transcriptional activity and/or binding in the presence of said agent, as compared to that in the absence thereof identifies said agent as a modulator of transcription;
c) administering said agent identified in b) to a non-human transgenic animal according to one of claims 1 to 7; and
d) measuring fat tissue growth and/or weight gain in the animal of step c) and comparing same with that of a control animal, not having been administered said agent, wherein a difference in fat tissue growth and/or weight gain of the animal of step c) as compared to that of the control animal identifies said agent as a modulator of fat tissue growth and/or weight gain in vivo.

29. The method of claim 28, where the agent is obtained from a library of compounds.

30. The method of claim 29, wherein the animal is a mammal.

31. The method of claim 30, wherein said mammal is a mouse or human.

32. A modulator of fat and/or glucose metabolism in vivo identified by any one of the methods of claims 28, 29, 30 or 31.

33. A method of treating and/or preventing obesity, comprising administering to an obese animal, or an animal susceptible of becoming obese, an agent which modulates the promoter activity of a promoter comprising a cis-acting element selected from the group consisting of:
i) an estrogen response element;
ii) TGA AGG TCA;
iii) AGG TCA NNN TGA CCT; and
iv) functional variants of l-iii)
wherein cis-acting element is capable of binding to ERRα.

34. The method of claim 33, wherein said agent reduces the level and/or activity of ERRα.

35. A method of determining whether an agent modulates obesity in an animal comprising:
a) providing a transcriptionally active preparation of ERRα or related factors and a DNA sequence comprising a promoter having a cis-acting sequence which modulates activity thereof by an interaction thereto of said ERRα and related factors;
b) measuring said transcriptional activity of said promoter or of a binding of at least ERRα or related factors to said cis-acting sequence in a presence and in an absence of an agent suspected of modulating the transcriptional activity of said promoter or the binding of said factors to said cis-acting sequence, thereby identifying an agent which modulates transcription of said promoter and wherein a difference in the transcriptional activity and/or binding in the presence of said agent, as compared to that in the absence thereof identifies said agent as a modulator of transcription;
c) administering said agent identified in b) to a non-human transgenic animal according to one of claims 1 to 7; and
d) assessing obesity in the animal of step c) and comparing same with that of a control animal, not having been administered said agent, wherein a difference in obesity of the animal of step c) as compared to that of the control animal identifies said agent as a modulator of obesity in vivo.

36. The method of claim 35, where the agent is obtained from a library of compounds.

37. The method of claim 36, wherein the animal is a mammal.

38. The method of claim 37, wherein said mammal is a mouse or human.

39. A modulator of glucose or fat metabolism in vivo identified by any one of the methods of claims 35, 36, 37 or 38.
